(19) United States
(12) Patent Application Publication
(10) Pub. No.: US 2023/0279473 A1
(43) Pub. Date: Sep. 7, 2023

(54) LATERAL FLOW NUCLEIC ACID ASSAY WITH INTEGRATED PORE-BASED DETECTION

(71) Applicants: THE REGENTS OF THE UNIVERSITY OF CALIFORNIA, Oakland, CA (US); ELECTRONUCLEICS, Placentia, CA (US)

(72) Inventors: Harold G. Monbouquette, Santa Monica, CA (US); Youngsam Bae, Placentia, CA (US); Zhenrong Zheng, Los Angeles, CA (US); Yan Cao, Los Angeles, CA (US); Jacob J. Schmidt, Sherman Oaks, CA (US)

(73) Assignees: THE REGENTS OF THE UNIVERSITY OF CALIFORNIA, Oakland, CA (US); ELECTRONUCLEICS, Placentia, CA (US)

(21) Appl. No.: 18/176,889

(22) Filed: Mar. 1, 2023

Related U.S. Application Data
(63) Continuation of application No. PCT/US2021/049529, filed on Sep. 8, 2021.
(60) Provisional application No. 63/075,669, filed on Sep. 8, 2020.

(51) Int. Cl. C12Q 1/6825; B01L 3/00

(52) U.S. Cl. CPC C12Q 1/6825; B01L 3/5023; B01L 2300/069; B01L 2300/0645; B01L 2300/0816; B01L 2200/0668

(57) ABSTRACT

An apparatus for a lateral flow nucleic acid assay with an integrated pore-based detector, which has the potential to detect pathogens, both microbial and viral, in aqueous samples in approximately 5 minutes or less without nucleic acid amplification or optical components. The detector is based on an electromechanical signal transduction mechanism that enables low-cost detection of DNA/RNA at ultralow concentrations (down to about 10 M to about 19 M). The scheme relies on the use of charge-neutral peptide nucleic acid (PNA) capture probe conjugated to polystyrene beads. The PNA-beads acquire substantial negative charge upon capture of target pathogenic DNA/RNA and become mobile in an electric field. Upon application of a bias voltage of around 1 V to 2 V, the PNA-beads with hybridized target are directed electrophoretically to a smaller diameter pore. Subsequent pore blockage results in a strong, sustained drop in measured ionic current through the pore.

### CROSS-REFERENCE TO RELATED APPLICATIONS

This application claims priority to, and is a 35 U.S.C. § 111(a) continuation of, PCT international application number PCT/US2021/049529 filed on Sep. 8, 2021, incorporated herein by reference in its entirety, which claims priority to, and the benefit of, U.S. provisional patent application Ser. No. 63/075,669 filed on Sep. 8, 2020, incorporated herein by reference in its entirety. Priority is claimed to each of the foregoing applications.

The above-referenced PCT international application was published as PCT International Publication No. WO 2022/056040 A1 on Mar. 17, 2022, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to detection of specific RNA or DNA fragments with complementary probe conjugated to charge neutral polystyrene beads, and more particularly to transverse flow detection of specific RNA or DNA fragments with complementary probe conjugated to charge neutral polystyrene beads.

### 2. Background Discussion

There is strong impetus for the conception and development of low-cost, accurate, and robust point-of-care (POC) nucleic acid (NA)-based diagnostic devices that give results in minutes. Most infectious disease diagnosis currently is accomplished by culturing methods that typically take days. POC immunoassays have been marketed for detection of pathogens, but these often have marginal sensitivities and specificities, while less common nucleic acid (NA)-based tests have ultralow limits of detection (LODs) and both sensitivity and specificity in the 90-99% range. A handful of POC NA-based tests are available for a few analytes including flu, respiratory syncytial virus (RSV), and Group A streptococcus.

The NA-based tests for other indications are clinical laboratory tests as in the cases of Neisseria gonorrhoeae (NG, gonorrhea) and Chlamydia trachomatis (CT, chlamydia) where the process of transporting samples to a lab, batching, testing, and returning results also typically takes days. Key points regarding the significance of the technology disclosed here are summarized below.

Current methods for most infectious disease diagnosis take a day or more, which can interfere with prompt administration of optimal therapies and counseling, result in reliance on undependable follow-up contacts, lead to inappropriate prescription of antibiotics, may cause extended patient suffering, and can contribute to high health care costs.

Rapid determination of the presence or absence of key pathogens in clinical samples, i.e., a qualitative test, usually is the paramount need including for flu, RSV, SARS-CoV-2, HIV, human papillomavirus (HPV), NG, CT, etc.

Methods with a sufficiently low detection limit based on NA detection without amplification are rare and generally require expensive reagents and/or complex analytical equipment.

Determination of the simple presence or absence (yes/no answer) of key pathogens in body fluid, i.e., a qualitative test, is of paramount need. Common pathogens, whose very presence at any level in body fluid is abnormal and is an indicator of infection, include SARS-CoV-2, Group A Streptococcus, Neisseria gonorrhoeae (NG), Chlamydia trachomatis (CT), influenza virus, and Bordetella pertussis (whooping cough).

Pathogen detection methods based on NA amplification have key disadvantages. All NA amplification-dependent devices must include subsystems for sample preparation (including pathogen lysis and NA purification), NA target amplification, and amplicon detection.

Generally, optical methods are used for amplicon detection, which necessitates the incorporation of optical components into the instrument with associated increased complexity and cost. Despite impressive advances in rapid methods for polymerase chain replication (PCR) cycling, the need for precise temperature control has driven many test developers to pursue isothermal amplification methods, but these methods still require primers, polymerases, and extensive NA purification to remove inhibitors of polymerases as well as reaction conditions that must be carefully controlled.

Broadly applicable, NA amplification-free, label-free, sequence-specific NA detection schemes are rare. Over the past 10 years or so, remarkable progress has been made in developing new approaches for amplification-free NA detection at clinically relevant concentrations in the single-digit attomolar (aM, 10⁻¹⁸ M) range and below. However, only a handful of these schemes do not require special labels other than an oligonucleotide complementary to the target NA. Also, nearly half require optics of some kind. The remaining approaches entail piezoelectrics, MALDI TOF MS (matrix-assisted laser desorption/ionization time-of-flight mass spectrometry), or various electrochemical techniques.

Of those schemes based on electrochemical detection, only one employs simple and inexpensive constant potential amperometry, yet still Pt nanoparticle labels are required. Ideally, an amplification-free NA sensor would involve just the selective oligonucleotide probe and eliminate the need for additional reagents, labels or complicated signal transduction technologies; however, the state of the art presented above suggests that sensing schemes that meet this ideal are rare.

Prior Work

The RNA/DNA detection device disclosed here is distinct from other nanopore-based NA sensing systems in that it is not a resistive-pulse sensor based on the work of Coulter (DeBlois R W, Bean C P. Counting and Sizing of Submicron Particles by the Resistive Pulse Technique. Review of Scientific Instruments. 1970; 41(7):909-16) where the conductance of an electrolyte-filled pore or channel is monitored as various analyte species traverse it. Rather, it is based on far simpler conductometric detection of large signals from long-lasting pore blockages.

The resistive-pulse approach is focused on precise measurement of small changes in nanopore current over short timescales (μs to ms) as analytes traverse a pore, whereas the device technology disclosed here intrinsically amplifies this signal into the nA-range and extends its duration indefinitely by relying on persistent pore blockages to signal the presence of analyte.

This approach greatly simplifies device electronics and readout, as described in Monbouquette, Harold and Schmidt, Jacob, PCT International Publication No. WO 2013/033647 A2 published on Mar. 7, 2013 and incorporated herein by reference in its entirety.

Nonspecifically bound NA rarely gives a persistent signal. Note that a “signal” is a persistent step reduction in ionic current that lasts several seconds or longer. Most control runs with non-complementary NA lead to no observable pore blockades; only some transient blockades (not lasting long enough to constitute a signal) are observed infrequently. Yet, it has been observed that incubation of the beads with non-complementary NA occasionally results in substantial nonspecific binding as noted by an increase in zeta potential from the single digit range to about 20 to about 30 mV. Thus, these beads with nonspecifically bound DNA are negatively charged and electrophoretically mobile, allowing them to be driven to the pore.

At the pore mouth, the electric field is sufficiently strong to remove the nonspecifically bound DNA from the bead, which causes a reduction in bead charge and electrophoretic mobility, enabling the opposing drag due to electroosmotic flow to exceed the electrophoretic force and carry the bead away from the pore. This electroosmotic flow arises from an opposing flux of positive counterions to fixed negative charges on the glass pore wall.

Experimental evidence, as well as results of many control studies, show that beads with only nonspecifically bound NA approach a pore mouth briefly and then are propelled away by the opposing electroosmotic flow despite possible mobility due to dielectrophoresis. The literature does not appear to disclose another NA-based diagnostic system with such an active system to avert false positives.

### BRIEF SUMMARY

This technology describes a lateral flow nucleic acid assay with an integrated pore-based detector and its method of use.

In one embodiment, the technology described in this disclosure comprises the integration of a glass chip harboring a thin glass membrane and pore with a lateral flow membrane and the use of magnetic polystyrene bead-PNA (peptide nucleic acid) conjugates to control bead location on the membrane and to position the beads in proximity to the glass chip for detection of bead-PNA conjugates with hybridized target nucleic acid. It should be noted that although magnetic polystyrene bead-PNA has been used in this embodiment, other magnetic substrates able to be conjugated with charge-neutral peptide nucleic acid (PNA) capture probes could also be used, including other charge neutral nucleic acid analogs.

The integrated device has the potential to detect pathogens, both microbial and viral, in aqueous samples in approximately 5 minutes or less without nucleic acid amplification or optical components. The detector relies on a novel electromechanical signal transduction mechanism that enables the low-cost, optics-free and amplification-free (e.g., no PCR) detection of DNA/RNA at ultralow concentration (as low as 10⁻¹⁹ M).

An important feature of the detector is the use of peptide nucleic acid (PNA) capture probes, which are uncharged polyamide analogs to NAs that share the same base chemistry. Since bead-PNA conjugates are designed to be charge neutral, they do not exhibit appreciable electrophoretic movement in the presence of a DC electric field. However, the substantial negative charge acquired upon capture of a target NA sequence makes the hybridized conjugate mobile.

Electrophoresis of the bead-PNA conjugate with hybridized target NA to the mouth of a smaller diameter glass pore causes a significant increase in pore resistance, thereby resulting in a persistent strong, sustained drop in measured ionic current. Nonspecifically bound NA is removed from the bead conjugate in the strong electric field in the pore mouth resulting in no sustained signal. Further, the opposing electroosmotic flow through the glass pore sweeps PNA-bead conjugates without hybridized target away from the pore mouth. In such a way, this simple conductometric device gives a highly selective (rarely observed false positives), binary response signaling the presence or absence of the target NA (and associated pathogen).

Diagnostic applications of the device and method include, but are not limited to: 1. any microbial or viral pathogen, e.g., SARS-CoV-2, flu, gonorrhea, chlamydia, RSV, Strep; 2. use in clinics, emergency rooms, or urgent care centers; 3. COVID-19 screening, e.g., dental appts, surgical appointments, job sites, small meetings; 4. home diagnostics; 5. food safety; and 6. hoof and mouth disease (cattle).

Additional military diagnostic applications may include, but are not limited to: diarrheal diseases; infected wound assay; biowarfare agents, e.g., anthrax, plague, etc.; and location specific pathogens, such as dengue or yellow fever.

The device and method are robust, low power (e.g. battery powered), likely handheld, and rapid (less than 5 minutes for detection).

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings. These drawings, which are not to scale, are for illustrative purposes only:

FIG. 1 is an overall diagram of the detection scheme for specific nucleic acids using PNA probe-conjugated, charge-neutral polystyrene beads.

FIG. 2A is a photograph of a 1 cm square borosilicate glass sample with a micromachined, submicron-thick membrane in the center.

FIG. 2B is a scanning electron micrograph (SEM) of the etched membrane of the nanopore of FIG. 2A, when viewed at an angle.

FIG. 2C is a SEM of a focused ion beam (FIB)-etched nanopore in the etched membrane of FIG. 2B, which may be used as a pore for nucleic acid detection as described herein.

FIG. 3A is a side view of the lateral flow nucleic acid assay with integrated pore-based detection.

FIG. 3B is an enlarged section of FIG. 3A, more clearly pointing out the geometries of the pore used in the integrated pore-based detection.

FIG. 3C is a top view of a polydimethylsiloxane (PDMS) top pattern deposited over a glass chip.

FIG. 4 is a view of the apparatus for detecting specific nucleic acids with probe-conjugated, charge-neutral polystyrene beads.

FIG. 5A and FIG. 5B provide an overall flowchart of a method for detecting specific nucleic acids with probe-conjugated, charge-neutral polystyrene beads.

FIG. 6 is a side view of the lateral flow strip assembly

FIG. 7A is a side view of the glass chip assembly.

FIG. 7B is a top view of a polydimethylsiloxane (PDMS) top pattern deposited over a glass chip.

FIG. 7C is a top a view of a polydimethylsiloxane (PDMS) bottom film deposited over a glass chip.

FIG. 8 is a side view of the whole system assembly.

FIG. 9 is a plot of the pore current as observed using a potentiostat to fix potential and measure ionic current through the pore.

### DETAILED DESCRIPTION

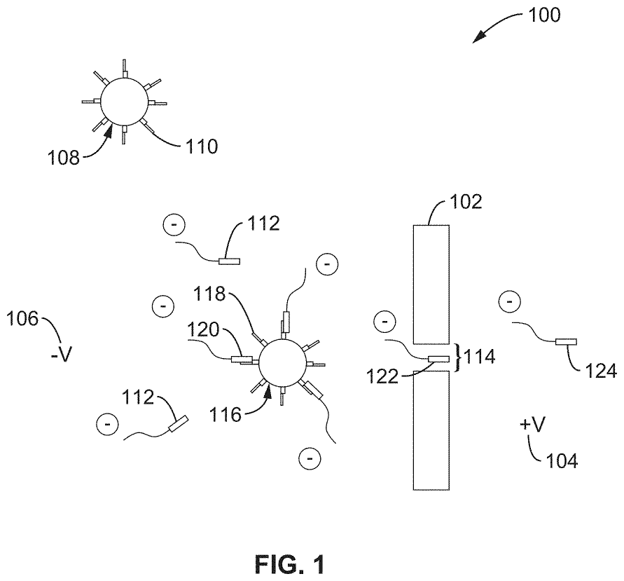

Refer now to FIG. 1, which is a diagram 100 of the operational characteristics of this apparatus. Initially, a membrane 102 is placed between a positive voltage V⁺ 104 and a negative voltage V⁻ 106.

The diagram shows polystyrene beads 108 with one or more covalently attached peptide nucleic acid (PNA) probes 110 complementary to single-stranded nucleic acid targets (DNA or RNA, 112) and a pore 114 through the glass membrane 102 that is of smaller diameter than the beads 108. The beads 108 are purchased with carboxyl groups on the surface that are used as attachment sites for an amine-terminated PNA probe 110. The particular beads 108 discussed in one embodiment are 820 nm in diameter.

Other bead and pore dimensions, and geometries, may work well. It has been found that submicron thick membranes worked best, but good performance may not be limited to this dimension if other materials are used. Furthermore, although a cylindrical pore 114 was a goal for fabrication, a substantially conical shape resulted. It is possible that other shapes for the pore 114 may also work, so long as the pore minimum dimension is sufficiently small that one or more hybridized beads can block ionic current through the pore 114, resulting in a drop of pore 114 current.

Initially, specific single-stranded nucleic acid targets (DNA or RNA, 112) are enveloped in a solution as unattached moieties. However, after some time, the polystyrene beads 108 with one or more covalently attached peptide nucleic acid (PNA) probes 110 complementary to specific single-stranded nucleic acid targets (DNA or RNA, 112) achieve hybridization with their targets. This is shown in the bonded polystyrene bead 116 to which the covalently attached peptide nucleic acid (PNA) probes 118 have, in some instances bonded 120 to the single-stranded nucleic acid targets (DNA or RNA, 112) in one or more places.

In this FIG. 1 diagram, it is seen that polystyrene bead 116 has hybridized to three instances of the single-stranded nucleic acid targets (DNA or RNA, 112). This bonding has created a net of many negative charges on the bonded polystyrene bead 116 corresponding to the length of the target, thereby enabling it to become electrophoretically motile (or electromotile) due to the imposed electric field between the applied positive voltage V⁺ 104 and a negative voltage V⁻ 106.

Normally, the charged single-stranded nucleic acid targets (DNA or RNA, 112) proceed 122 without interruption through the pore 114 in the membrane 102 since the pore 114 is much larger. Therefore, the charged single-stranded nucleic acid targets (DNA or RNA, 122) pass through the much larger pore 114 without causing an appreciable disruption in the ionic current through the pore 114.

In this diagram, single-stranded nucleic acid targets (DNA or RNA, 124) have already passed through the pore 114 in the membrane 102.

PNA is an uncharged nucleic acid analog. Any remaining carboxyl groups are capped first with amine-terminated polyethylene glycol (PEG) and then with ethanolamine. It is important that PNA be conjugated on the beads at an optimal surface density. Remaining carboxyl groups on the bead surface must be capped. Here, polyethylene glycol (PEG) is used to help prevent bead aggregation. Ethanolamine is used to cap any remaining carboxyl groups and is necessary to achieve near electroneutrality. After these bead modification steps, the beads have low single-digit, negative mV zeta potential (and are essentially neutral) and are not appreciably mobile in a moderate electric field.

However, RNA and DNA 112 carry substantial negative charges, and when target RNA or DNA hybridizes 120 to the PNA on the modified beads, as shown on the bonded polystyrene bead 116, the complex carries sufficient negative charge to be mobile in the imposed electric field (V⁻ 106 to V⁺ 104).

When the PNA-beads with hybridized target, resulting in the bonded polystyrene bead 116, approaches the pore 114 opening, an appreciable, sustained deflection of ionic current occurs. This sustained reduction in ionic current is termed “persistent”.

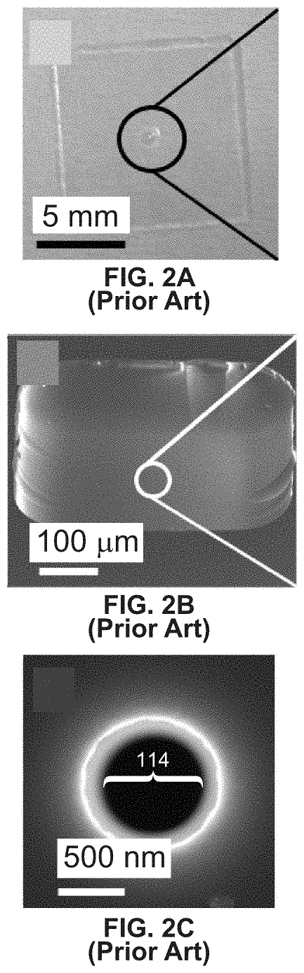

Refer now to FIG. 2A through FIG. 2C, all of which are prior art taken from Koo B, Yorita A M, Schmidt J J, Monbouquette H G. “Amplification-free, sequence-specific 16S rRNA detection at 1 aM.” Lab Chip. 018; 18(15):2291-9. doi: 10.1039/C8LC00452H.

FIG. 2A is a photograph of a 1 cm square borosilicate glass sample with a micromachined nanopore in the center of a thinned etched region.

FIG. 2B is a scanning electron micrograph (SEM) of the etched membrane of the nanopore of FIG. 2A, when viewed at an angle.

FIG. 2C is a SEM of a focused ion beam (FIB) created nanopore in the etched membrane of FIG. 2B. Such a nanopore may be used as the pore 114 of FIG. 1.

Here, borosilicate glass has been used because of its wide availability in the scientific community. However, the pore material could in principle be any material with a substantial surface concentration of fixed negative charges so that an electroosmotic flow could be developed to help prevent false positive test results. Additionally, composites of two or more materials could also be used.

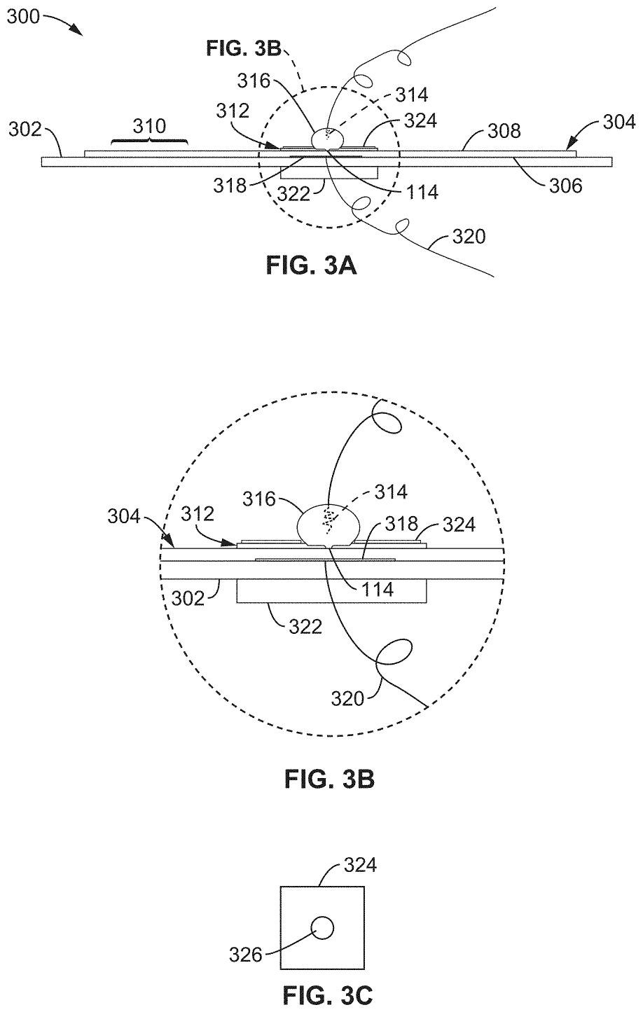

Refer now to FIG. 3A, FIG. 3B, and FIG. 3C.

FIG. 3A is a side view 300 of one embodiment of a lateral flow nucleic acid assay with integrated pore-based detection. A glass substrate, such as a borosilicate glass microscope slide, is used as a substrate 302. Upon the glass substrate 302 is placed a membrane 304, which has a bottom side 306 and a top side 308. On one lateral side of the membrane 304 is a sample loading area 310.

A glass chip 312 comprises a fabricated micro- or nano-pore 114 as previously described above in FIG. 1. This pore 114 is difficult to view in this drawing, since it is about 500 nm in diameter. The glass chip 312 is attached to the membrane 304 on the top side 308, and conductively coupled to a platinum electrode 314 on the other side through the use of a conductive buffer 316 droplet.

These glass chips 312 will be incorporated into single-use assay cartridges containing the PNA-beads and the process fluidics.

Between the membrane 304, bottom side 306, and the substrate 302 is also emplaced a platinum foil electrode 318 to which a conductive wire 320 is attached. The platinum foil electrode 318 and the platinum electrode 314 are situated in such a way as to conduct a sensible current through the glass chip 312 as ions pass through the pore 114.

Although a foil electrode 318 is shown here, other electrode configurations could be substituted, such as a simple wire, a patterned wire, or even a thin film conductor deposited directly on the substrate 302.

In operation, a sample is loaded onto the sample loading area 310, where the membrane 304 transports the sample laterally across the glass chip 312 via capillary action of the membrane 304, and more importantly, in proximity to the pore 114. Such capillary based membranes 304 may be nitrocellulose-based, glass fiber-based, or other material essentially nonreactive to the materials used in practicing this invention.

One example of the membrane 304 would be the Fusion 5 membrane product by Cytiva (unbacked such that both sides are water permeable). Such a membrane 304 has a large enough effective pore size such that either magnetic or non-magnetic PNA-beads can move through it. With the Fusion membrane 304, a separate sample loading area 310 (composed of a different material) would be unnecessary, but a separate sample pad could be used. If larger liquid samples are used, an additional absorption pad may be added downstream of the glass detector to absorb excess liquid, thereby facilitating flow along the lateral flow membrane.

FIG. 3B is an enlarged section of the side view of the lateral flow nucleic acid assay with integrated pore-based detection of FIG. 3A. This is enlarged so that the minute details of the glass chip 312 and pore 114 may be better appreciated.

FIG. 3C is a top view of a polydimethylsiloxane (PDMS) top pattern deposited over the glass chip 312.

Now refer to FIG. 3A, FIG. 3B, and FIG. 3C. Micromachined nanopore glass chips 312 facilitate high-throughput manufacturing, more straightforward interfacing to POC microfluidic devices, and the production of low-cost devices. Such glass chips 312 have been developed using a MEMS (MicroElectroMechanical systems) process to create submicron thick borosilicate glass membranes with 100 nanometer- to micron-scale pores 114.

Cartridges containing such glass chips 312 will likely be inserted into a handheld base unit containing inexpensive electronics, a display, and wireless communications.

In another embodiment of the lateral flow nucleic acid assay with integrated pore-based detector 300, a magnet 322 is used to maintain a position of polystyrene beads comprising magnetite, thereby having ferromagnetic properties, and thus attracted to the magnet 322. Note that other magnetic materials may be used to render the polystyrene beads ferromagnetic. The magnet 322, which can be a neodymium magnet, another permanent magnet, or an electromagnet, is used to hold the magnetic PNA-beads in place while sample is drawn over the beads to effect hybridization.

It should be noted that in FIG. 3C, a top pattern 324 of polydimethylsiloxane (PDMS) is seen. This pattern is deposited on a top surface of the glass chip 312 so as to better keep the conductive buffer 316 droplet from spreading away from the platinum electrode 314. This is better accomplished via the circular opening 326 situated over the pore 114.

The target nucleic acid hybridizes to the PNA-beads when a sample, introduced at the sample loading area 310, flows over them. Next the magnet 322 is removed so that the beads with hybridized target can move toward the glass chip and block the pore 114. Typically, the sample is moved by capillary action of the membrane 304 by addition of a chaser fluid, that acts to “flush” the target toward the pore 114.

### DISCUSSION

This technology represents a potentially significant advance in NA detection enabling a device that is low cost, low power (e.g., battery powered), portable, compact, rapid and robust. As part of a device, the detector is ideally integrated with an overall process flow for sample collection, cell lysis, NA extraction, and target NA hybridization to PNA probes on magnetic beads.

Sample collection and lysis will likely be conducted simultaneously and separately in a syringe pre-loaded with lysis buffer. The sample (e.g., urine, blood) will be drawn into the syringe, lysis (i.e., chemical disruption of microbial cell envelopes or disruption of viral capsid) will occur in about a minute. Subsequently, several drops of lysed sample will be deposited onto the sample pad area of the assay device through a submicron filter (about 0.1 μm pore size) attached on the syringe. The filter is likely necessary to remove particulate matter that, if negatively charged, could cause pore blockage and result in a false positive signal.

PNA-beads are deposited previously on the membrane 304 in a position very near to the glass chip 312 detector or directly beneath it. The glass chip 312 may be deposited on the membrane 304 by any means that enable it to be attached in a state where the glass membrane 304 is wetted without any trapped air bubbles on either side.

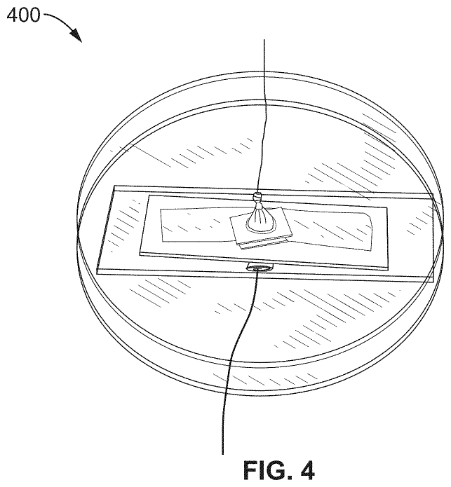

Refer now to FIG. 4, which is a view 400 of the detector of the apparatus for detecting specific nucleic acids with probe conjugated charge neutral polystyrene beads. This is a line drawing taken from a photograph of the device of FIG. 3A through FIG. 3B during actual operation.

Example Procedure

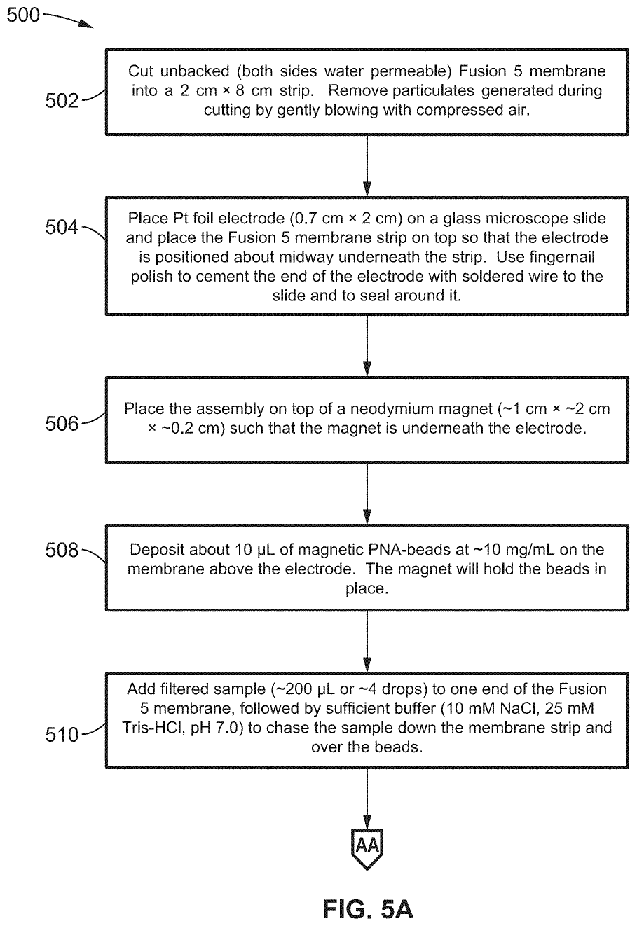

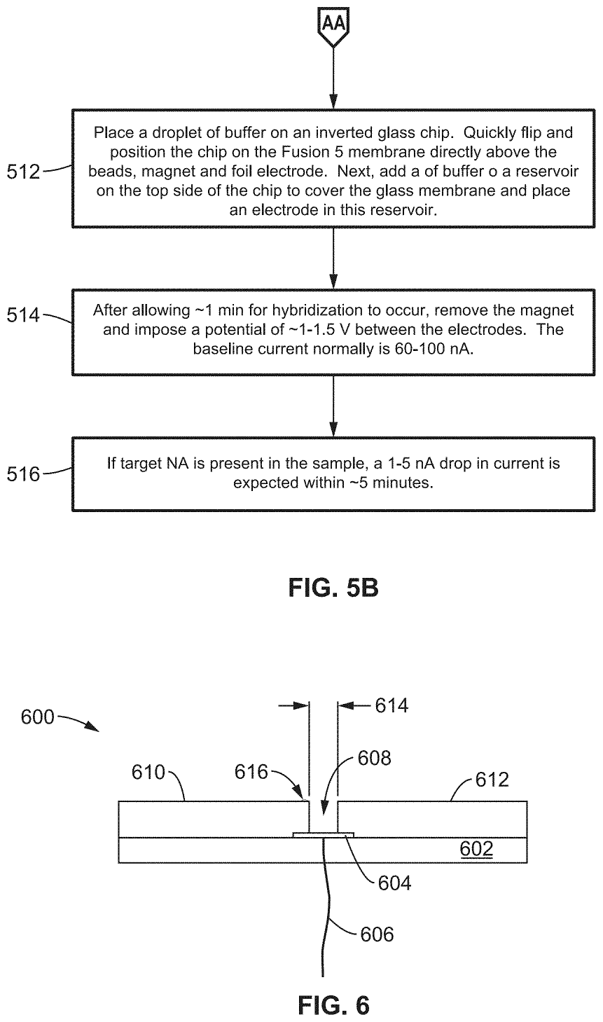

Refer now to FIG. 5A and FIG. 5B, which are a flowchart 500 of a method for detecting specific nucleic acids with probe conjugated charge neutral polystyrene beads.

At 502:
Unbacked (both sides water permeable) Fusion 5 membrane is cut into a 2 cm×8 cm strip, and particulates generated during cutting are removed by gently blowing with compressed air

At 504:
The Pt foil electrode (0.7 cm×2 cm) is placed on a glass microscope slide and the Fusion 5 membrane strip is placed on top so that the electrode is positioned about midway underneath the strip. Fingernail polish is used to cement the end of the electrode with soldered wire to the slide and to seal around it.

At 506:
The assembly is placed on top of a neodymium magnet (about 1 cm×about 2 cm×about 0.2 cm) such that the magnet is underneath the electrode.

At 508:
About 10 μL of magnetic PNA-beads at about 10 mg/mL are deposited on the membrane above the electrode. The magnet should hold the beads in place.

At 510:
Filtered sample (about 200 μL or about 4 drops) is added to one end of the Fusion 5 membrane (in the sample loading area), followed by sufficient buffer (10 mM NaCl, 25 mM Tris-HCl, pH 7.0) to chase the sample down the membrane strip and over the PNA beads.

At 512:
A droplet of buffer is placed on an inverted glass chip. The chip is then quickly flipped and positioned on the Fusion 5 membrane directly above the PNA beads, magnet and foil electrode. Next, a drop of buffer is added to a reservoir on the top side of the chip to cover the glass membrane and an electrode is placed in this reservoir.

At 514:
After allowing about 1 min for hybridization to occur, the magnet is removed, and a potential of about 1 V to about 1.5 V is imposed between the electrodes. The baseline current normally is about 60 nA to about 100 nA.

At 516:
If target NA was present in the sample, a nA-range drop in current is expected within about 5 minutes.

Further Developments

Demonstration of this method and lateral flow apparatus has only been conducted at relatively high E. coli sample concentration of about 10,000 CFU/m L. In future work, it is likely that a limit of detection of at least 10 CFU/mL using this setup could be demonstrated, which still would be orders of magnitude higher than what may be possible given that a detection limit of about 100 zM rRNA has been demonstrated with the detector alone, which corresponds to about 1 CFU/100 mL.

Conceptually, a commercial device resembling that described above is envisioned. However, it is likely that the Fusion 5 membrane strip will be incorporated dry within a single-use cartridge. The dry membrane will likely have predeposited buffer salts in the sample pad area to control pH as well as predeposited, magnetic or non-magnetic PNA-beads. The magnet likely will be an electromagnet incorporated into the base unit. An absorbent pad at the opposite end from the sample pad likely will be used to draw fluid through the Fusion 5 membrane.

The aspect of the interfacing of the glass chip with the Fusion 5 membrane is unclear in a manufacturable cartridge. One approach would be to house the glass chip in a wet state (no gas bubbles) sealed from the rest of the cartridge until some point after the cartridge is inserted into the base unit.

The electronics maintain the potential across the glass membrane at about 1 V to about 2 V while monitoring current. A drop in the pore transit current on the screen may be seen, which is a detection event. Sometimes a double drop is observed that may be due to multiple beads clustering around the pore

While developing this system, nucleic acid had often been extracted using a commercially available kit like that described in the Koo paper cited previously. However, it has also been shown (but as yet not published) that sample lysis at pH about 10 for about 1 min followed by about 0.1 μm filtration and neutralization appears adequate. A syringe-type sampling device is being developed that would draw up about 1 mL of sample (urine, blood, saliva, buffer that a sampling swab was swished in) into a chamber with preloaded, concentrated high pH buffer (or dry buffer salts). After waiting about 1 min, the syringe would be depressed, but the outflowing lysed sample would go through an about 0.1 μm filter and several drops of this lysed and filtered sample would be deposited on the lateral flow strip. In the preferred arrangement, there would be dried neutralization buffer on the strip to neutralize the pH prior to the lysed sample flowing over the PNA-beads. Alternatively, another chamber could be added to the syringe sampling device for neutralization to occur prior to filtration.

A Further Embodiment of the Lateral Flow Nucleic Acid Assay with Integrated Pore-Based Detection System

1. Introduction

This embodiment is also based on a persistent pore blockage by conjugated PNA capture probes. However, this embodiment has no requirement for the pore blockage polystyrene bead to be magnetic.

2. Lateral Flow Strip Assembly

Refer now to FIG. 6, which is a side view of the lateral flow strip assembly 600.

The assembly procedural steps are as follows:

Cut A-4 size Fusion 5 membrane sheet from Cytiva into 1.5 cm×3 cm strips.

Use a paper cutter to cut the Cytiva backing card into 1.5 cm×8 cm pieces. This becomes the backing 602 for the assembly.

Cut the Pt-foil 604 into 2 mm×1 cm small strips and solder a wire 606 on the end to make a Pt-foil electrode 608.

Peel off the film on the backing card and attach a soldered Pt-foil electrode 608 in the middle of the backing 602 card.

Attach two Fusion 5 membrane pieces, a loading side 610, and an absorbing side 612, on top of the edges of the Pt-foil electrode 608 and the backing 602. A less than 1 mm gap 614 is left between these two Fusion 5 membrane pieces to expose the Pt-foil electrode underneath. This assembled card is known as the lateral flow strip assembly 600.

PNA-modified beads are placed at a point 616 on the loading side 610 of the lateral flow strip assembly 600, as further detailed below.

Mark one side of the chip to be the loading side 610.

Wash PNA/PEG/ethanolamine-modified polystyrene beads with hybridization buffer (10 mM NaCl, 25 mM Tris-HCL, pH 7, 1% Tween 20).

Concentrate the beads by centrifuge filtration and load the beads next to the gap 614, at the loading point 616.

Before the beads get dried, apply vibration force to the beads by holding the lateral flow membrane strip to the wall of a bath sonicator. This step helps prevent bead aggregation.

Let the beads dry.

3. Glass Chip Assembly

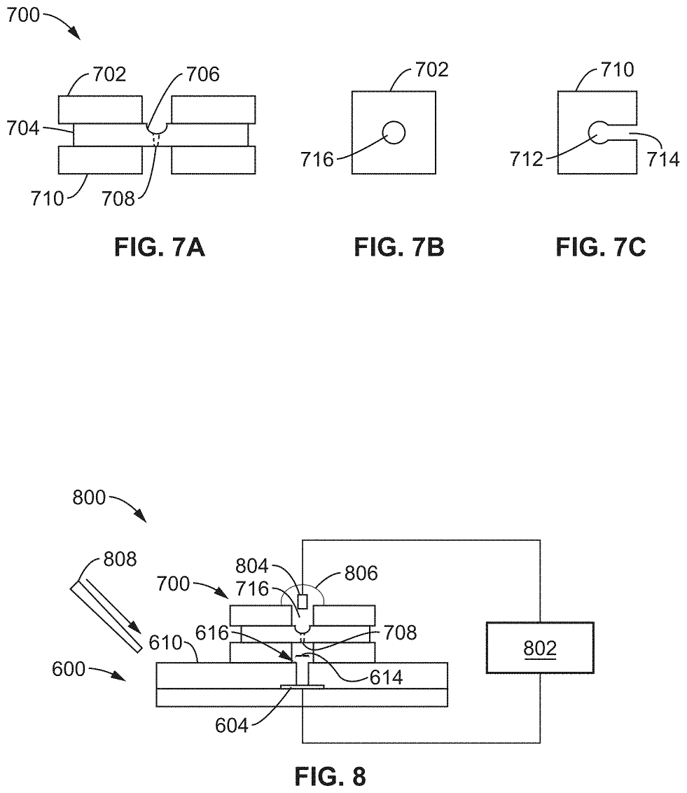

Refer now to FIG. 7A through FIG. 7C. FIG. 7A is a side view of the glass chip assembly 700. FIG. 7B is a view of a polydimethylsiloxane (PDMS) top pattern 702 deposited over a glass chip 704. This glass chip 704 has been previously etched to form a thinned region 706 less than 1 μm thick, and subsequently FIB processed to manufacture a nanopore 708.

As shown in FIG. 7A, the glass chip 704 with an about 1 μm to about 800 nm diameter “nanopore” 708, which must be smaller than bead diameter used, is sandwiched in the middle between two PDMS O-ring shaped films 702 and 710. Cellophane tape (e.g., Scotch tape) is used to briefly remove the dust on the PDMS films 702, 710, and this will ensure good attachment to the glass chip 704.

Now turning to FIG. 7C, we see a view of a polydimethylsiloxane (PDMS) bottom film 710 deposited over a glass chip 704.

The bottom PDMS film 710 is about 0.3 mm thick and with a circular opening 712 to expose the nanopore 708. A channel 714 is formed all the way from the edge to the circular opening 712. This design enables air to escape when the underlying Fusion 5 membrane is wetted (see below) and tends to prevent the formation of bubbles. The top PDMS film 702 has about 1 mm thickness and it also has a circular opening 716 to expose the nanopore. This circular opening 716 acts as a buffer reservoir for the top electrode that is used in detection see below).

4. Whole System Assembly

Refer now to FIG. 6, FIG. 7A, and FIG. 8. FIG. 8 is a side view of the whole system assembly 800.

Attach the lateral flow strip assembly 600 to a potentiostat 802 by using one Ag/AgCl electrode 804 as both counter and reference electrode, while using the Pt-foil 604 electrode as the working electrode.

Put the glass chip assembly 700 on the top of the lateral flow strip assembly 600. Position the nanopore 708 right on top of the gap 614 in the lateral flow strip assembly 600.

Put a droplet 806 of hybridization buffer in the circular opening 716 of the PDMS top pattern 702 of the glass chip assembly 700.

Gently lower the Ag/AgCl electrode 804 mentioned above into the top buffer reservoir droplet 804 in the PDMS top pattern 702, also mentioned above, to establish an electrical connection through the nanopore 708 in the glass chip assembly 700.

5. Detection

Deposit about 400 μL of the testing sample 808 onto the loading side 610 of the lateral flow strip assembly 600. Due to capillary flow, the liquid sample will flow over PNA-modified beads placed at the point 616 so that the target RNA or DNA in the sample will hybridize with the PNA probe on the modified beads.

The beads move more slowly in the Fusion 5 membrane than the fluid but at least some get carried into the gap below the pore in the glass chip. Fluid will pass through the gap 614 and to the absorbing side 612 of the Fusion 5 membrane. Fluid also will fill the opening in the lower PDMS O-ring shaped film 710 below the glass chip 704, and air will escape through the channel 714 in the lower PDMS O-ring shaped film 710 so that bubbles will not form.

It should be noted that this gap might not have to be completely free of any material. It just has to be sufficiently open (high porosity, large enough pore size) such that the hybridized PNA-beads move well in it, and movement to block the pore 114 is not obstructed.

During this process, the power to the potentiostat 802 is turned on and data is collected using software on a computer.

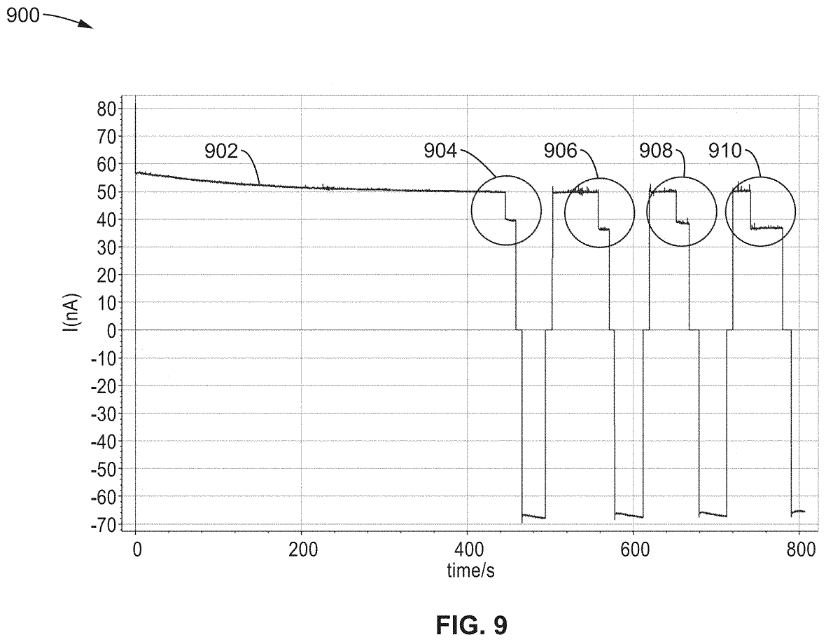

Refer now to FIG. 9, which is a plot 900 of the pore 708 current as observed by the potentiostat 802.

For reference, the “potentiostat” can actually be a very simple device used to fix the transpore voltage and monitor current. In practice, it can be as simple as a battery-supplied voltage source and current monitor.

A stable baseline 902 current appears. For a positive test, after a few minutes, a sustained drop in the current occurs due to the PNA-beads with hybridized target nucleic acid blocking the nanopore 708, and this is taken as the detection signal. For a negative test, there is just a stable baseline current, with no current drops observed.

This FIG. 9 shows typical successful detection data, where detection of aM E. coli 16S rRNA in buffer is achieved. Four example detection signals are circled 904, 906, 908, and 910. The electric field polarity was reversed after the first three detection signals 904, 906, and 908, and then returned after the first three events. A return to baseline 902 is observed followed by a repeated signal 906, 908, 910.

It should be noted that in this embodiment, the magnet 322 of FIG. 3A is no longer required, as there is no need for holding of magnetic PNA-beads in place as in another embodiment.

From the description herein, it will be appreciated that the present disclosure encompasses multiple implementations of the technology which include, but are not limited to, the following:

Integration of (i) a glass chip with a thin glass membrane and pore with (ii) a lateral flow membrane, and the use of magnetic bead-PNA conjugates to control bead location on the membrane and to position the beads in proximity to the glass chip for detection of bead-PNA conjugates with hybridized target nucleic acid.

A lateral flow assay apparatus comprising a glass chip with an upper electrode, a lateral flow membrane, and a lower electrode, wherein the glass chip is integrated with the lateral flow membrane.

An apparatus for detecting specific nucleic acids, comprising: (a) a lateral flow membrane having a top side, a bottom side, a loading side, and an absorbing side; (b) a pore in contact with the top side of the lateral flow membrane; (c) a bottom electrode disposed on the bottom side of the lateral flow membrane; and (d) a top electrode disposed above the pore, the top electrode immersed in buffer; (e) wherein addition of buffer to wet the lateral flow membrane on the loading side causes a lateral flow of the buffer to pass by the pore en route to the absorbing side; (f) wherein the buffer is deposited sufficiently so as to conduct a current that may be detected between the top electrode and the bottom electrode; and (g) wherein the current passes through the pore upon application of a voltage between the top electrode and the bottom electrode.

The apparatus of any preceding or following implementation, wherein the pore is of a substantially cylindrical to conical shape with a smallest diameter of about 500 nm, and a typical height of less than about 1 μm.

The apparatus of any preceding or following implementation, wherein the pore substantially comprises borosilicate glass.

The apparatus of any preceding or following implementation, further comprising: (a) a glass chip assembly, comprising: (i) an etched portion of the borosilicate glass typically less than or equal to about 1 μm in thickness; (ii) wherein the pore is disposed within the etched portion; and (iii) a polydimethylsiloxane (PDMS) top pattern deposited over the borosilicate glass comprising a circular opening centered over the pore, and on a side opposite from the pore.

The apparatus of any preceding or following implementation, further comprising: (a) one or more charge-neutral peptide nucleic acid (PNA) capture probes conjugated to polystyrene beads; (b) wherein the PNA capture probe is designed to capture a target pathogenic DNA/RNA.

The apparatus of any preceding or following implementation, wherein a diameter of the pore is less than the diameter of the polystyrene beads.

The apparatus of any preceding or following implementation, further comprising: (a) a magnet adjacent to a deposition point of the polystyrene beads; (b) wherein the polystyrene beads comprise magnetic material in part; and (c) wherein the magnet attracts and retains the polystyrene beads.

An apparatus for detecting specific nucleic acids, comprising: (a) a lateral flow strip assembly comprising: (1) a backing: (2) a loading side disposed on the backing, (3) an absorbing side disposed on the backing, (4) an electrode disposed on the backing in electrical contact with both the loading side and absorbing side; (5) a gap disposed between the loading side and the absorbing side, the gap disposed above the electrode; and (6) one or more peptide nucleic acid (PNA) beads deposited at a location on the loading side; (b) a glass chip assembly, comprising: (1) a glass chip having a top side and a bottom side; (2) an etched region less than about 1 μm thick disposed on the bottom of the glass chip; (3) a nanopore disposed in the etched region of the glass chip; (4) a polydimethylsiloxane (PDMS) top shape with a first circular opening disposed on the top side of the glass chip; and (5) a PDMS bottom shape with a second circular opening disposed on the bottom side of the glass chip, wherein this shape comprises an open channel from the second circular opening to an edge of the shape; and (c) a whole system assembly, comprising: (1) the lateral flow strip assembly attached to the glass chip assembly; (2) wherein the gap in the lateral flow assembly aligns with the nanopore of the glass chip assembly; and (3) a potentiostat connected to the foil electrode and to a Ag/AgCl electrode positioned above the nanopore. Although the Ag/AgCl electrode has been used here, an alternate material could be used that could simultaneously act as both a reference and counter electrode.

The apparatus of any preceding or following implementation, further comprising: (a) a droplet of hybridization buffer disposed in the circular opening of the PDMS top pattern of the glass chip assembly; (b) wherein the Ag/AgCl electrode is immersed at one end in the droplet.

The apparatus of any preceding or following implementation, wherein the potentiostat measures a current that passes through the nanopore.

An apparatus for detecting specific nucleic acids, comprising: (a) a glass chip with a thin glass membrane and pore; (b) a lateral flow membrane in contact with the pore; and (c) a magnetic bead-PNA conjugate; (d) wherein the magnetic bead-PNA conjugate location is controlled on the membrane via a magnet; and (e) wherein the magnetic bead-PNA conjugate is positioned in proximity to the glass chip pore for detection of bead-PNA conjugates with hybridized target nucleic acid.

A method for detecting a target nucleic acid (NA), the method comprising: (a) cutting unbacked Fusion 5 membrane into a strip, and removing particulates generated during cutting; (b) placing a foil electrode on a glass microscope slide, and placing the Fusion 5 membrane strip on top of the electrode so that the electrode is positioned about midway underneath the strip; (c) placing the glass slide on top of a neodymium magnet such that the magnet is positioned underneath the electrode; (d) depositing magnetic PNA-beads on the membrane at a position above the electrode, wherein the magnet should hold the beads in place; (e) adding a lysed and filtered sample to one end of the Fusion 5 membrane, followed by sufficient buffer to chase the sample down the membrane strip and over the beads; (f) placing a droplet of buffer on an inverted glass chip; (g) flipping the glass chip and positioning the glass chip on the Fusion 5 membrane directly above the beads, magnet and foil electrode; (h) adding a drop of buffer to a reservoir on the top side of the glass chip and placing an upper electrode in the reservoir; and (i) waiting for hybridization to occur, removing the magnet, and applying an electric potential between the electrodes; (j) wherein if a target NA is present in the sample, a drop in electric current is observed.

A method for detecting a target nucleic acid (NA), the method comprising: (a) providing a lateral flow membrane comprising a top side, a bottom side, a loading side, and an absorbing side; (b) providing a pore in contact with the lateral flow membrane; (c) providing a bottom electrode disposed on the bottom side of the lateral flow membrane; (d) providing a top electrode disposed above the pore, the top electrode immersed in buffer; and (e) dispensing buffer to wet the lateral flow membrane on the loading side thereby causing a lateral flow of the buffer to pass by the pore en route to the absorbing side; (f) wherein the buffer is deposited sufficiently so as to conduct a current that may be detected between the top electrode and the bottom electrode; and (g) wherein the current passes through the pore upon application of a voltage between the top electrode and the bottom electrode.

A method for detecting a target nucleic acid (NA), the method comprising: (a) providing a charge-neutral peptide nucleic acid (PNA) capture probe conjugated to polystyrene beads; (b) providing a pore in contact with a lateral flow membrane; (c) lysing a sample; (d) filtering the lysed sample; (e) laterally flowing the lysed and filtered sample adjacent to the pore; (f) applying a voltage across the pore; (g) detecting an ionic current passing through the pore; and (h) detecting a specific nucleic acid through a persistent drop in ionic current passing through the pore.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, upper and lower, left and right, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly, the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for detecting specific nucleic acids, comprising:
(a) a lateral flow membrane having a top side, a bottom side, a loading side, and an absorbing side;
(b) a pore in contact with the top side of the lateral flow membrane;
(c) a bottom electrode disposed on the bottom side of the lateral flow membrane; and
(d) a top electrode disposed above the pore, the top electrode immersed in buffer;
(e) wherein addition of buffer to wet the lateral flow membrane on the loading side causes a lateral flow of the buffer to pass by the pore en route to the absorbing side;
(f) wherein the buffer is deposited sufficiently so as to conduct a current that may be detected between the top electrode and the bottom electrode; and
(g) wherein the current passes through the pore upon application of a voltage between the top electrode and the bottom electrode.

2. The apparatus of claim 1, wherein the pore is of a substantially cylindrical to conical shape with a smallest diameter of about 500 nm, and a height of less than about 1 μm.

3. The apparatus of claim 2, wherein the pore substantially comprises borosilicate glass.

4. The apparatus of claim 3, further comprising:
(a) a glass chip assembly, comprising:
(i) an etched portion of the borosilicate glass less than or equal to about 1 μm in thickness;
(ii) wherein the pore is disposed within the etched portion; and
(iii) a polydimethylsiloxane (PDMS) top pattern deposited over the borosilicate glass comprising a circular opening centered over the pore, and on a side opposite from the pore.

5. The apparatus of claim 1, further comprising:
(a) one or more charge-neutral peptide nucleic acid (PNA) capture probes conjugated to polystyrene beads;
(b) wherein the PNA capture probe is designed to capture a target pathogenic DNA/RNA.

6. The apparatus of claim 5, wherein a diameter of the pore is less than the diameter of the polystyrene beads.

7. The apparatus of claim 6, further comprising:
(a) a magnet adjacent to a deposition point of the polystyrene beads;
(b) wherein the polystyrene beads comprise magnetite in part; and
(c) wherein the magnet attracts and retains the polystyrene beads.

8. An apparatus for detecting specific nucleic acids, comprising:
(a) a lateral flow strip assembly comprising:
(1) a backing:
(2) a loading side disposed on the backing,
(3) an absorbing side disposed on the backing,
(4) an electrode disposed on the backing in electrical contact with both the loading side and absorbing side;
(5) a gap disposed between the loading side and the absorbing side, the gap disposed above the electrode; and
(6) one or more peptide nucleic acid (PNA) beads deposited at a location on the loading side;
(b) a glass chip assembly, comprising:
(1) a glass chip having a top side and a bottom side;
(2) an etched region less than 1 μm thick disposed on the bottom of the glass chip; and
(3) a nanopore disposed in the etched region of the glass chip;
(4) a polydimethylsiloxane (PDMS) top shape with a first circular opening disposed on the top side of the glass chip; and
(5) a PDMS bottom shape with a second circular opening disposed on the bottom side of the glass chip, wherein this shape comprises an open channel from the second circular opening to an edge of the shape; and
(c) a whole system assembly, comprising:
(1) the lateral flow strip assembly attached to the glass chip assembly;
(2) wherein the gap in the lateral flow assembly aligns with the nanopore of the glass chip assembly; and
(3) a potentiostat connected to the foil electrode and to an Ag/AgCl electrode positioned above the nanopore.

9. The apparatus for detecting specific nucleic acids of claim 8, further comprising:
(a) a droplet of hybridization buffer disposed in the circular opening of the PDMS top pattern of the glass chip assembly;
(b) wherein the Ag/AgCl electrode is immersed at one end in the droplet.

10. The apparatus for detecting specific nucleic acids of claim 9, wherein the potentiostat measures a current that passes through the nanopore.

11. An apparatus for detecting specific nucleic acids, comprising:
(a) a glass chip with a thin glass membrane and pore;
(b) a lateral flow membrane in contact with the pore; and
(c) a magnetic bead-PNA conjugate;
(d) wherein the magnetic bead-PNA conjugate location is controlled on the membrane via a magnet; and
(e) wherein the magnetic bead-PNA conjugate is positioned in proximity to the glass chip pore for detection of bead-PNA conjugates with hybridized target nucleic acid.

12. A lateral flow assay apparatus comprising a glass chip with an upper electrode, a lateral flow membrane, and a lower electrode, wherein the glass chip is integrated with the lateral flow membrane.

13. A method for detecting a target nucleic acid (NA), the method comprising:
(a) cutting unbacked Fusion 5 membrane into a strip, and removing particulates generated during cutting;
(b) placing a foil electrode on a glass microscope slide, and placing the Fusion 5 membrane strip on top of the electrode so that the electrode is positioned about midway underneath the strip;
(c) placing the glass slide on top of a neodymium magnet such that the magnet is positioned underneath the electrode;
(d) depositing magnetic PNA-beads on the membrane at a position above the electrode, wherein the magnet should hold the beads in place;
(e) adding a lysed and filtered sample to one end of the Fusion 5 membrane, followed by sufficient buffer to chase the sample down the membrane strip and over the beads;
(f) placing a droplet of buffer on an inverted glass chip;
(g) flipping the glass chip and positioning the glass chip on the Fusion 5 membrane directly above the beads, magnet and foil electrode;
(h) adding a drop of buffer to a reservoir on the top side of the glass chip and placing an upper electrode in the reservoir; and
(i) waiting for hybridization to occur, removing the magnet, and applying an electric potential between the electrodes;
(j) wherein if a target NA is present in the sample, a drop in electric current is observed.

14. A method for detecting a target nucleic acid (NA), the method comprising:
(a) providing a lateral flow membrane comprising a top side, a bottom side, a loading side, and an absorbing side;
(b) providing a pore in contact with the lateral flow membrane;
(c) providing a bottom electrode disposed on the bottom side of the lateral flow membrane;
(d) providing a top electrode disposed above the pore, the top electrode immersed in buffer; and
(e) dispensing buffer to wet the lateral flow membrane on the loading side thereby causing a lateral flow of the buffer to pass by the pore en route to the absorbing side;
(f) wherein the buffer is deposited sufficiently so as to conduct a current that may be detected between the top electrode and the bottom electrode; and
(g) wherein the current passes through the pore upon application of a voltage between the top electrode and the bottom electrode.

15. A method for detecting a target nucleic acid (NA), the method comprising:
(a) providing a charge-neutral peptide nucleic acid (PNA) capture probe conjugated to polystyrene beads;
(b) providing a pore in contact with a lateral flow membrane;
(c) lysing a sample;
(d) filtering the lysed sample;
(e) laterally flowing the lysed and filtered sample adjacent to the pore;
(f) applying a voltage across the pore;
(g) detecting an ionic current passing through the pore; and
(h) detecting a specific nucleic acid through a persistent drop in ionic current passing through the pore.
